(19) United States
(12) Patent Application Publication
(10) Pub. No.: US 2006/0166353 A1
(43) Pub. Date: Jul. 27, 2006

(54) Process for realishing a biomorphic, stereolithographed phantom, which is multicompartmental and suitable for multanalytical examinations, and relevant device

(76) Inventors: Bruno Alfano, Napoli (IT); Anna Prinster, Napoli (IT); Mario Quantarelli, Napoli (IT)

Correspondence Address: YOUNG & THOMPSON, 745 SOUTH 23RD STREET, 2ND FLOOR, ARLINGTON, VA, 22202, US

(21) Appl. No.: 10/528,825

(22) PCT Filed: Sep. 22, 2003
(86) PCT No.: PCT/IT03/00564

§ 371 Date: Jan. 4, 2006

(30) Foreign Application Priority Data
Sep. 25, 2002 (IT) RM2002A000477

(51) Int. Cl. C12M 1/34

(52) U.S. Cl. 435/287.8

(57) ABSTRACT

A process for preparing digital images for realising a biomorphic multicompartmental phantom, includes a phase A.1 of acquisition of images of the organ belonging to the analysed living being, forming a volumetric image defined by voxels, a phase A.2 of identification of tissues and/or tissue liquids and a phase B of selection of at least three tissues and/or tissue liquids, a phase C.1 for verifying the adjacency of the voxels belonging to each single tissue or tissue liquid, a phase C.3 for preparing an image presenting the surfaces of the volumes defined in phase C.1 according to a sub-phase C.3.2 which determines a number of surfaces equal to the number of tissues, and a phase C.3.3 which assigns a thickness to the surfaces.

The present invention relates to a process for realising a biomorphic, stereolithographed phantom, which is multicompartmental and suitable for multianalytical examinations, and to the relevant device as well.

More in detail, the invention concerns a process for producing, in particular through stereolithography, a biomorphic phantom, for instance representing the brain of superior primates, which presents several compartments fillable with different liquid solutions or mixtures and which appears to belong to the biological form from which it is derived to the researches through the emission tomography and the transmission one, and to other techniques as nuclear magnetic resonance as well.

Generally, the phantoms are objects used in the context of imaging diagnostics for testing the performance of several apparatus. Generally, they are designed for a determined category of equipments such as the emission tomography, both the Positron Emission Tomography (PET) and Single Photon Emission Tomography (SPECT), the Transmission Topography (CT), Magnetic Resonance Imaging (MRI), the Computerised Axial Tomography (CAT) or Computed Tomography (CT).

The phantoms may be of geometric or anthropomorphic type.

The geometric ones, generally simpler, are used for carrying out measurements of specific characteristics such as spatial resolution or homogeneity of response.

The anthropomorphic phantoms are the ones simulating form and composition of a portion of the human body or of a part of it, in the sense that, if subject to a specific diagnostic examination, they produce images similar to the ones produced by the human body subject to the same diagnostic examination. These phantoms are generally used for quantifying the error made in carrying out, through diagnostic studies, measurements of chemical-physical parameters on a patient, such as for instance radioisotope concentrations and volumetric measurements. This type of check is generally the more accurate the more the phantom approximates the real situation.

To the knowledge of the inventors, the phantoms of anthropomorphic type realised so far are:

the 2D or 3D brain phantom by Hoffman for use in nuclear medicine;

an anthropomorphic phantom of torso for use in nuclear medicine;

CIRS 3D brain phantom for localization for use in operations;

Striatal Phantom for Use in PET/SPECT by Alderson;

CROBOT of torso for use in colonoscopy; and

NEUROBOT, a brain phantom for localization for operations;

the phantom realised by Tanikawa et al. for optical tomography

The phantom by Hoffman is a series of plastic discs which form a fillable chamber simulating the brain wherein the grey matter is completely filled with the solution containing the tracer, while the solid layers, reducing the volume which may be occupied by the solution, which simulate the behaviour of the white matter in nuclear medicine (with a ratio of 4:1 between the tracer concentration for the grey matter and the one for the white matter), The phantom does not itself represent a human brain, but It simulates its behaviour so that the images of nuclear medicine seem the ones of a real brain, instead the images of Magnetic Resonance or of CT do not appear so.

The CIRS 3D brain phantom is a cast of the scalp realised in a material which may be displayed on radiographic, CT and MRI images. The phantom simulates the bone of the cranium and the flesh surrounding it and it may be used for localization problems during surgical operations. The phantom is not multicompartmental, it cannot be used in nuclear medicine (MN) and its use is strictly limited to the application for which it has been realised.

The Striatal Phantom is anthropomorphic and multicompartmental, but the represented compartments are made of the caudate nuclei, the putamen and the rest of the brain, with no separation among white matter, grey matter and cerebrospinal fluid. It may be used in MN, CT and MRI but only for imaging the striatum.

The CROBOT phantom, still under prototyping, provides for the construction of a hollow human torso internally having a structure similar to the colon in order to be capable to simulate operations in colonoscopy, while the NEUROBOT phantom should represent a brain for leading a surgeon during certain operations.

The phantom realised by Tanikawa et al. for optical tomography provides a phantom with internal free spaces through which liquid can flow to simulate dynamically some brain functions.

Each one of the phantoms listed above is intended for a well specific application, that is for setting machines for a limited set of analytical methods often applied only to specific organs or tissues.

This limitation has enabled, from time to time, the avoidance of technical and practical problems, by selecting the most favourable technique of realisation to a specific case.

Consequently, no one of the single aforesaid phantoms may be suitable for setting all the PET, SPECT, MRI, MN, CT, CAT techniques or methods, simulating any type of tissue or even any set of tissues, and leading to an anthropomorphic representation of the concerned organs or tissues.

If any phantom among the ones listed above is taken, and it is used in another application, it does not work or it gives only approximate results not suitable for testing the analysing machines.

Even the phantom realised by Tanikawa et al. for optical tomography has severe limits, in that the internal free spaces have to be fabricated by hands: it cannot be realistic and its fabrication is cumbersome.

The aforesaid limitations actually come from the lack of an automated process which enables to pass from images of living beings to the effective production of the phantom and which comprises a processing which minimises the information of said images in order to save the production resources and hence to minimise the product cost, keeping in any case the universality of the produced phantom.

It is therefore an object of the present invention an automated process for generating three-dimensional maps of a multicompartmental and anthropomorphic phantom for use in researches which are conducted with different procedures, even multiple ones, by simulating any group of organic tissues.

It is still a specific object of the present invention a phantom which is produced starting from the maps which are obtained through the process according to the present invention.

It is therefore subject matter of this invention a process for preparing a three-dimensional digital image for realising a biomorphic multicompartmental phantom, representing at least one organ and/or at least one system belonging to a living being, comprising a first phase A.1 of acquisition of images or “sequences” of the organ or of the system belonging to the living being, according to predefined acquisition parameters, forming a volumetric image defined by voxels, further comprising a phase A.2 of identification of tissues and/or tissue liquids and a phase B of selection of

at least three of said tissues and/or tissue liquids, the process being characterised in that it comprises the following phases:

C.1 verifying the adjacency of the voxels, so that each tissue or tissue liquid defines a connected volume representing the tissue or tissue liquid itself;

C.3 preparing an image presenting the surfaces of the volumes defined in phase C.1 according to the following sub-phases:

C.3.2 determining a number of surfaces equal to the number of tissues, such that they result internal to one another, even if partially tangent, said surfaces being the convolution of the surfaces of one or more volumes defined in phase C.1, said surfaces giving, by addition or subtraction, all the surfaces corresponding to the tissues or tissue liquids selected in phase B;

C.3.3 assigning a thickness to said surfaces, so that in the portions wherein two or more surfaces are tangent to one another the thickness is assigned to only one surface, the set of said thicknesses forming a connected volume.

Preferably according to the invention, phase C.1 comprises the following sub-phases:

C.1.1 selecting a voxel from the set of voxels forming the whole acquired volume;

C.1.2 comparing the selected voxel with a neighbourhood of six voxels which are connected to it through one face;

C.1.3 if another voxel of the same type (belonging to the same tissue or tissue liquid) does exist in said neighbourhood, examining the neighbourhood of this one, and so on recursively;

C.1.4 if during phase C.1.3 an island of one or more connected voxels of the type selected in phase C.1.1 is identified, which is surrounded by one or more volumes of voxels of other types, carrying out the following sub-phase:

C.1.4.1 if said island has size smaller than a predetermined threshold, assigning the voxels of said island to the tissue which is most represented in a region including the island.

Additionally according to the invention, the process may further comprise, after phase C.1.4.1, a phase C.1.4.2 wherein, according to the method of the previous phases, the existence of islands having size larger than said threshold is verified and, in the positive, one of the following sub-phases is alternatively carried out:

reassign the island to one of said tissues or tissue liquids;

connecting the island, through a channel, to one of said tissues or tissue liquids.

This is done for avoiding possible problems related to the selection of a too small threshold or to possible (even if unlikely) segmentation errors.

Preferably according to the invention, the process further comprises a phase C.2 of smoothing the images in the three dimensions.

Also, it is preferable according to the invention that phase B of the process comprises the following phases:

B.1 eliminating all the tissues except a predetermined set of tissues;

B.2 filling the holes by assigning the corresponding voxels to at least one tissue of the predetermined set.

According to the invention, the process may include carrying out, before phase C.3.2, the following phase:

C.3.1 transforming the vector representation of the voxels into the vector representation of the surfaces separating the several tissues.

Preferably according to the invention, the organ of the living being, the images of which are acquired in phase A.1, is the brain of a superior primate.

Still more preferably according to the invention, the organ of the living being, the images of which are acquired in phase A.1, is the brain of a human being.

Advantageously according to the invention, during phase A.1 it is acquired a number of axial images ranging from 60 to 300, with layers having thickness ranging from 1 to 4 mm and with spacing from a centre to another one ranging from 0,5 to 2 mm, said images representing axial sections of the brain.

Advantageously according to the invention, said images which are acquired are MRI images.

Preferably according to the invention, the T1-w and PD-T2-w sequences are acquired for each localization of layer.

Also, preferably according to the invention, said at least three tissues or tissue liquids selected in phase B are the grey matter, the white matter and the encephalorachidian liquid.

According to the invention, during phase C.3.2 a first surface containing the white matter plus the grey matter, a second surface containing only the grey matter, and a third surface representing the cranium surface may be selected, the volume containing the encephalorachidian liquid and the volume containing only the white matter being obtained by subtraction between said surfaces.

Advantageously according to the invention, phase B has a phase B.3 in which the definition of the tissues in the images under processing is corrected and in which the definition and the form of the basal ganglia of the brain may be improved.

Preferably according to the invention, the image obtained from phase C.3.3 is modified so as to create channels entering the compartments/chambers corresponding to the selected tissues or tissue liquids, said channels being used for filling and emptying the phantom.

It is further specific subject matter of the present invention, an apparatus for processing images starting from images of an organ of a living being, characterised in that it automatically carries out in a configurable mode phases A.1 and A.2, and also phases B and C.

It is further specific subject matter of the present invention, a computer program characterised in that it comprises code means adapted to execute, when running on a computer, the process according to what just said.

It is still specific subject matter of the present invention, a memory medium readable by a computer, storing a program, characterised in that the program is the computer program according to what aforesaid.

It is finally specific subject matter of the present invention, a biomorphic multicompartmental phantom, suitable for multianalytical examinations, characterised in that it is produced through a rapid prototyping device using the images processed according to the process according to what aforesaid, the surfaces having thickness being made of solid synthetic matter and the volumes representing the various tissues and/or tissue liquids being left empty and so forming several fillable compartments.

Preferably according to the invention, the rapid prototyping device is a stereolithographer.

According to the invention, said compartments are filled with water or solutions containing radioisotopes, for its use in Nuclear Medicine.

Still according to the invention, said compartments are filled with solutions of contrast media or paramagnetic ions, for use in Computerised Axial Tomography and Magnetic Resonance.

Furthermore according to the invention, said compartments are filled with aqueous solutions of nickel and/or manganese and/or gadolinium.

The invention will be now described, by way of illustration and not by way of limitation, according to its preferred embodiments, by particularly referring to the figures of the enclosed drawings, in which:

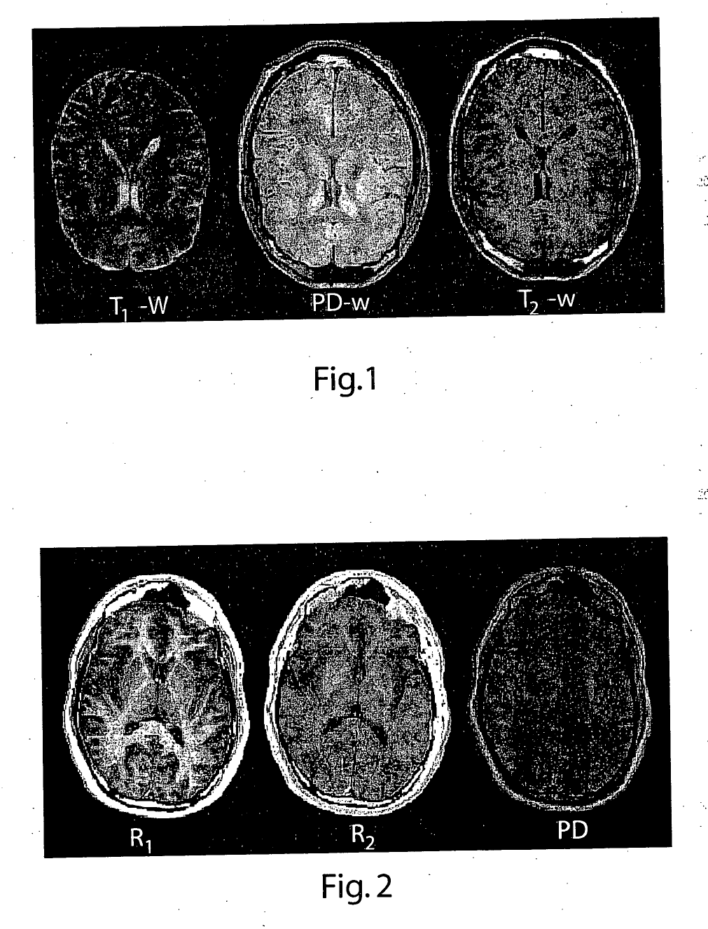

FIG. 1 shows three MRI images of a living brain section;

FIG. 2 shows other three images of a living brain section of a brain organ which represent three chemical-physical parameters (R1, R″ and PD) which are recalculated starting from the MRI images;

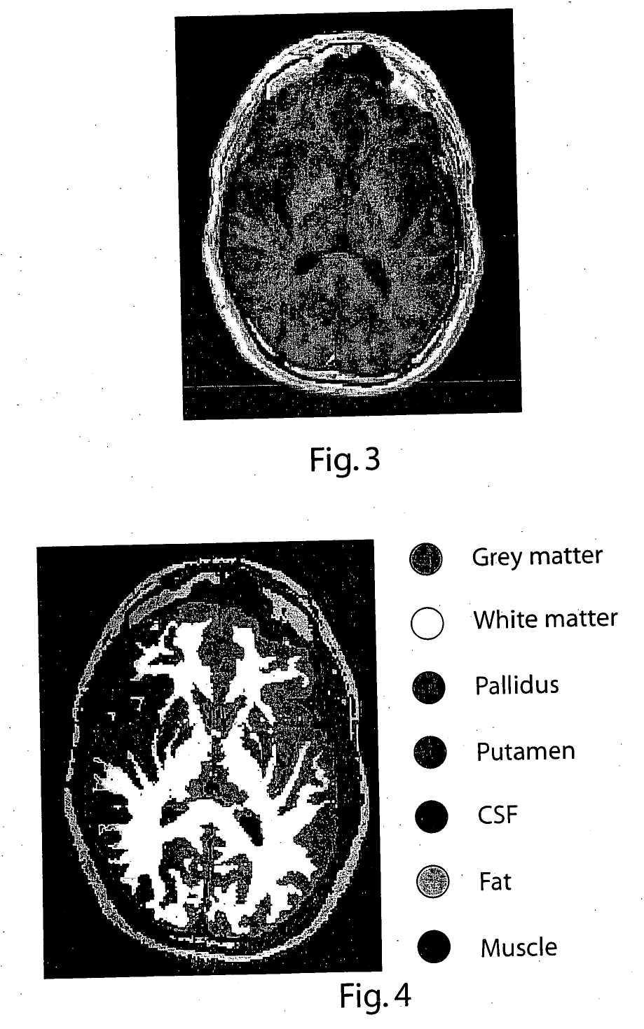

FIG. 3 shows the merge of the images of FIG. 2, having assigned the primary colours (red green and blue) to each image and having added up the three components;

FIG. 4 shows a segmented image of a brain section, i.e. the image of FIG. 3, with the indication of the identified tissues;

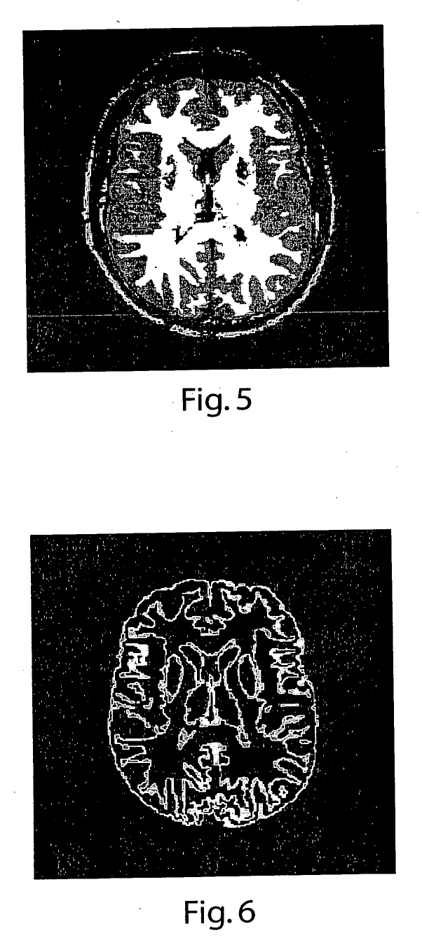

FIG. 5 shows a segmented image of a brain cross section of an healthy subject which is obtained through a MRI scan;

FIG. 6 shows the section, corresponding to FIG. 5, of the separating surfaces between grey matter (GM), white matter (WM) and cerebrospinal fluid (CSF);

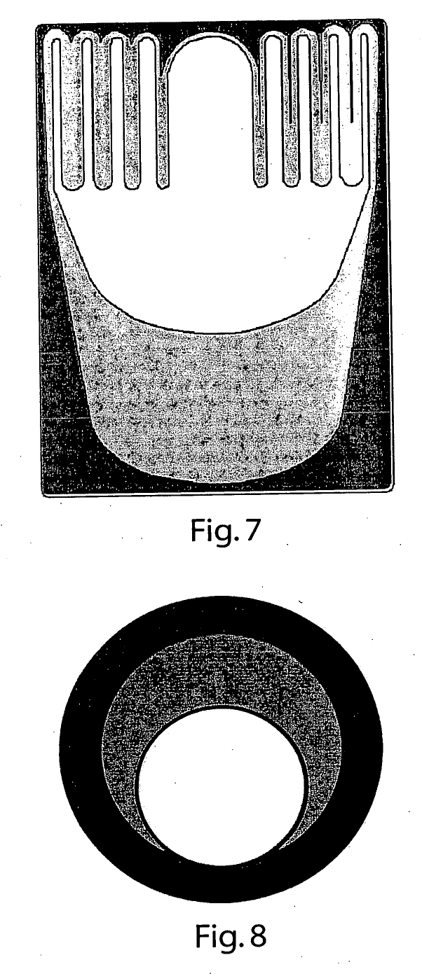

FIG. 7 shows a simplified brain model with simple topology having areas or more generally volumes wherein each volume (except the largest one) is defined by a surface which is completely internal to another surface of another volume, where a volume is tangent to only one other volume at most;

FIG. 8 shows a brain model with complex topology, having areas or more generally volumes wherein some volumes are tangent to several volumes;

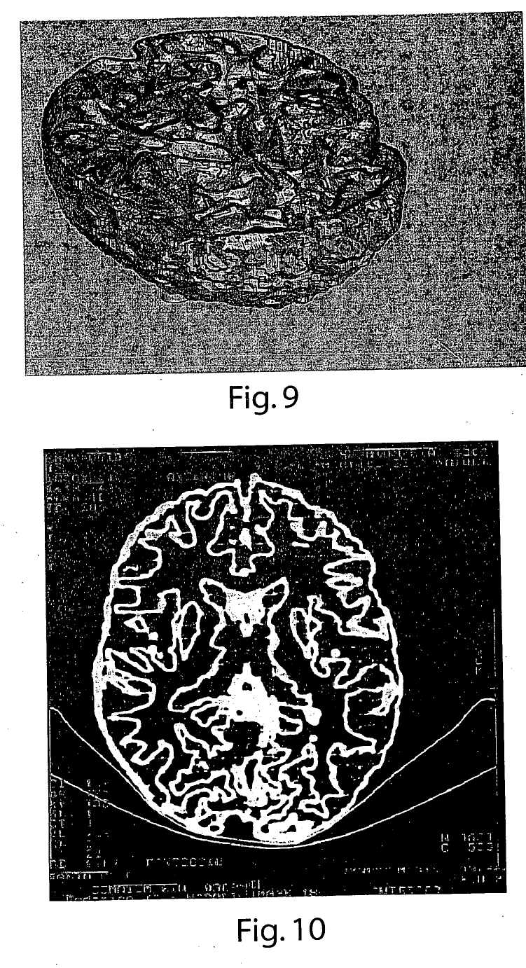

FIG. 9 shows a section of the section volumetric three-dimensional drawing of the phantom according to the present invention;

FIG. 10 shows a section which is obtained with a CT scan of the phantom constructed on the basis of the MRI data at about the level of the section of FIG. 5;

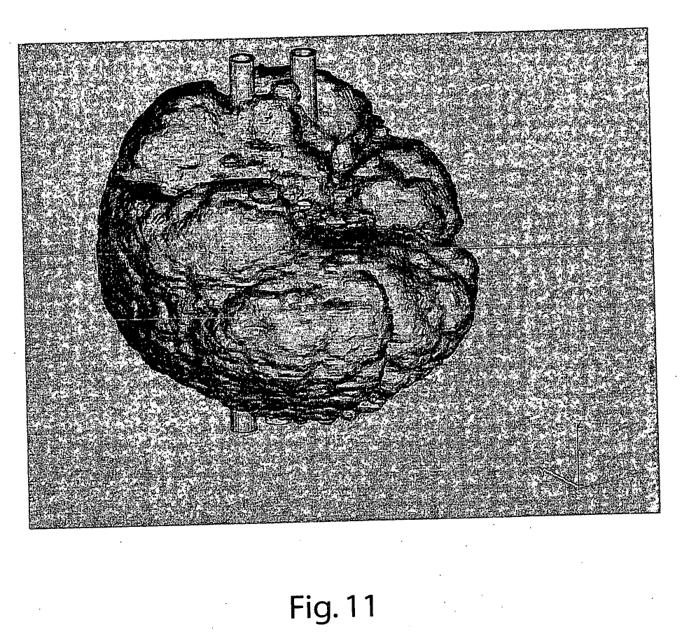

FIG. 11 shows the external surface of the phantom to which inlet and breather channels for aqueous solutions have been added;

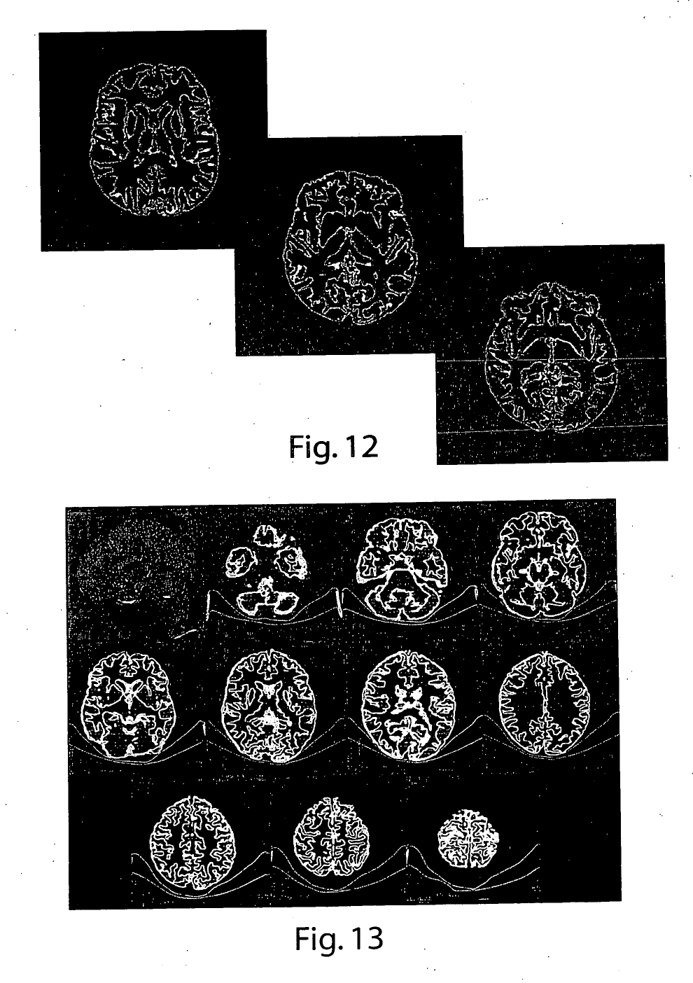

FIG. 12 shows three processed images of a brain section, showing the outlines of some tissues;

FIG. 13 shows images as in FIG. 12, but taken from the phantom according to the present invention;

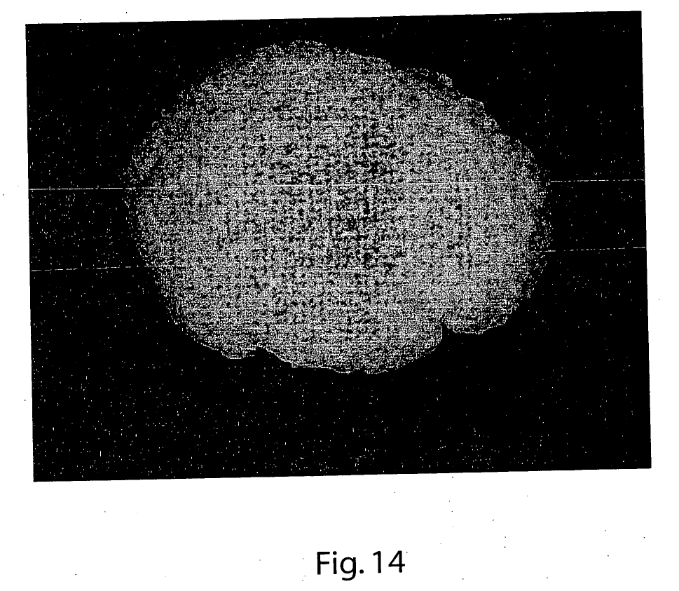

FIG. 14 shows a photograph from the outside of a prototype of the phantom according to the invention.

In the following of the description same references will be used to indicate alike elements in the Figures.

In the following example the process according to the invention will be considered in an application for obtaining a phantom of human brain, but it is clear that the same process may be applied to any other organ, either human or not (in this sense it is possible to say “biomorphic” phantom). It is also clear that the same process may be applied to several organs following the same steps and that it may hence be applied to a whole living being.

The process for processing the three-dimensional topology of the phantom according to the present invention has three main phases A, B and C.

The first phase A comprises a first sub-phase of acquiring images of the brain, the so-called “sequence”, according to predefined acquisition parameters.

The sequences, of the type shown in FIG. 1, are in such a number to carry out a scan of the whole brain organ, and usually contemporaneously all the voxels (which are the three-dimensional equivalent of the pixels), which form the brain volume, are defined. In fact, the images obtained for instance through MRI are grouped so as to form a volume with isotropic voxel having size equal to 1 mm.

Subsequently, a sub-phase of identification of the tissues, also called “segmentation”, is carried out. To this end, it is preferable to use the method disclosed by patents U.S. Pat. No. 5,486,763 and EP 0.603.323.

Several tones of grey, which are a function of both the acquisition parameters and the chemical-physical parameters to be purposely detected for identifying the tissues, are assigned to the voxels, as it has been made in FIG. 2 starting from the images of FIG. 1.

Starting from these sequences, it is possible to calculate, for each voxel, the chemical-physical parameters which generally are a function of the relaxation velocity parameters R1 and R2 (inverse of the relaxation times T1 and T2), and PD parameter (“proton density”), thus obtaining maps showing the distribution of each one of them inside the brain.

Moreover, the values of these parameters may control a RGB assignment for obtaining coloured maps, such as the one of FIG. 3.

Starting from these coloured maps, called images of Quantitative Magnetic Colour Imaging (QMCI), segmented maps are calculated, that is the tissues are classified, obtaining an image the colours of which are obtained as a weighted mean of the colours of said maps, such as the one of FIG. 4.

The above segmentation comprises the use of a known procedure wherein a voxel is represented in the parameter space and it is assigned to a tissue. Hence, in this phase it is also easy to establish possible pathologies, to be considered or not for further processing the images and for producing the phantom. In particular, the automated segmentation of pathological white matter (multiple sclerosis and leukaraiosis plaques) may be provided.

All the above has been, for instance, carried out for prototyping, starting from a MRI acquisition of a brain in its neurovegetative configuration of an healthy subject (NV), according to the following specifications:

image of a 36-years-old male healthy subject acquired through a Marconi 1.5T scanner;

5 sets of 30 axial images with 3 mm thick layers and 1 mm spacing from a centre to another one;

T1-w and PD-T2-w sequences for each localization of layer, such as for instance the ones of FIG. 1.

An example of the set of acquisition parameters of said images is:

series T1: 15/600 ms (TE/TR),

series PD-T2: 15/90/2300 ms (TE1/TE2/TR),

total acquisition time: about 20 minutes.

The so obtained MRI images represent axial sections of the brain.

In the second phase B, the acquired images are processed for selecting the tissues of interest, i.e. the volumes of organic substance which will form as many compartments in the phantom.

To this end, the preferred embodiment of the present invention comprises the following sub-phases of the phase B:

elimination of all the tissue except the white matter and the grey matter, the volume containing the CSF being obtained by subtraction with the cranium surface which is placed around the phantom of brain;

correction of the map for defining the basal ganglia (small formations inside the brain), since the automated segmentation of very small structures may sometimes be not satisfactory;

elimination of the system of blood vessels, by filling with white or grey matter.

It is not superfluous now to recall that the elimination of tissues or systems is carried out only on purpose of simplification, but it is in any way possible to take into account all the systems/tissues in order to produce a complex and very realistic phantom.

Moreover, the just listed second and third sub-phases may be inverted one another.

In the last phase C, the encoded images resulting from phase B are further processed for obtaining final maps, intended for controlling a phantom producing machine.

Such a machine is preferably a rapid prototyping device, still more preferably a stereolithographer.

The phase C comprises at first a sub-phase of verification of the adjacency of the voxels, verifying that each compartment/tissue is closed and inside completely connected, and contemporaneously eliminating the noise and the tissue islands smaller than a certain threshold.

This sub-phase comes from a well defined problem. In fact, the segmentation procedure may leave a trace of noise in the images, whereby some voxels which are erroneously assigned to a tissue may result isolated within another one. For instance, a tissue which enters another one forming a filament thinner than the voxel size will be segmented with a series of voxels which are separated or connected through only one corner.

In order to eliminate these “islands” it has been developed an automated procedure comparing each voxel with the neighbourhood of six voxels which are connected to it through one face. If another voxel of the same type (belonging to the same tissue) exists in this set, then a neighbourhood of this voxel is examined, and so on recursively. If the island of interconnected voxels is smaller than a predetermined threshold, these voxels are assigned to the tissue which is most represented in a neighbourhood of the island.

The same assignment method has been used for eliminating the holes left by the vessels irrorating the tissues. Once the islands corresponding to a predetermined threshold are eliminated, it is verified that there are no larger islands. In case such large islands are found, it is decided, on the basis of the known anatomy of the brain, whether they may exist in their proportions and locations.

If this cannot be, they have been evidently produced by the segmentation and by the subsequent elimination of the isolated voxels, which has deleted thin channels interconnecting said large islands.

In this case, it is possible to manually operate for reconstructing said lost connection. In this regard, it is however necessary to say that during experimentation this has never occurred and that the aforesaid test on the large islands is used as a preventive verification.

This verification uses, for each tissue, a routine written in the Interactive Data Language (IDL) that, starting from a voxel, looks for all the voxels of the same type which are connected to it within a 3D volume.

Subsequently, the following sub-phases are provided:

smoothing the images in the three dimension, since the compartment has to be fillable and hence the walls have not to be excessively ragged, in order to avoid air microbubbles;

extracting the outlines of the WM and GM chambers, and creating outlines having defined thickness;

adding the channels entering the WM and GM compartments/chambers for filling and emptying the phantom.

The first one of the just listed sub-phases may also be carried out before the phase preceding the present one, and this is preferable. The smoothing is necessary in order to flatten a little bit the outlines of the tissues taking into account the resolution limits of the stereolithography system.

At the end of this processing, that is at the end of the phase of creation of the entrance channels, it has been obtained a volume wherein the only represented tissues, i.e. the white matter, the grey matter and the CSF, form three compartments singularly connected and contiguous among them.

The sub-phase extracting the outlines of the WM and GM chambers actually comprises two sub-phases:

passing from the bit-map type representation for voxel to the vector representation of the surfaces separating the several tissues;

extracting the external surfaces of the white matter and of the white plus grey matters.

With the first of these sub-phases, the passage from the image of FIG. 5 to the one of FIG. 6 is for example operated.

In the second one, it is necessary to pass from the processed volume containing the representation of the three tissues in binary form to a representation of the walls which separates the several compartments and which has to be realised, in particular through stereolithography.

Since the stereolithography machine materialises the volume defined by one or more closed surfaces, vectorially represented (in STL format), in order to realise the very thin walls defining the tissue compartments it is necessary: extracting from each compartment represented in binary form the surface defining it; representing in an unique space the aforesaid surfaces; doubling each surface by creating another one (otherwise it is possible to create two surfaces starting from the separation one) which is internal to it and spaced a constant minimum distance apart assuring the solidity of the wall. In the case of the three considered brain compartments it is also fundamentally important to minimise the overlapping of the walls, since the spatial coincidence of two vector surfaces is never perfect and thus generates a swelling of the resulting wall.

Since the morphology of the brain compartments is more complex than the one providing for a volume internal to another one as in FIG. 7, a topological representation of the three compartments effectively studied in this example (see FIG. 8) may clarify the problem. In the figure the white substance is represented in white, the grey substance in grey and the CSF in azure. The white substance abuts on the grey one and the CSF; the grey one abuts on the white one and the CSF; the CSF abuts on both and the cranium. Since in the preferred embodiment it has been decided to separately realise the cranium (external container of the phantom), the problem is reduced to optimise the realisation only of the brain parenchyma (grey matter and white matter, the CSF being consequently defined by the additional surface of the cranium, as specified).

Passing through the representation of FIG. 8 it may be verified that the optimal solution is realising the walls defining the compartment of the white substance and the parenchyma compartment (grey plus white substances). In fact, this solution limits the zone having overlapped walls to the only boundary zone between white substance and CSF, a very limited zone wherein the wall thickness is not critical.

The extraction of the external surfaces of the white matter and of the white plus grey ones is hence the solution to the technical problem of using a stereolithographer for producing volumes with external surfaces not internal to one another. The process is also valid when the volumes to be defined are more than three.

At this point, images of the outlines of chambers/tissues, including the thicknesses of the surfaces separating the chambers, have been obtained, as in FIG. 9.

Once the phantom is realised starting from this image, it will appear as anthropomorphic to researches normally used for patients, as it may be verified by comparing the image of FIG. 10 with the one of FIG. 6.

Finally, during the phase of creation of the entrance channels, the numerical images are modified in order to form artificial WM and GM channels for filling the compartments (in case of the brain, the preferred location is the top part in order to optimise the filling), and also auxiliary breather channels for the emptying, as shown in FIG. 11, at a location opposite to the filling channels.

Lastly, a further action which is necessary for purely practical purposes, and which form a further sub-phase of the phase C, is the insertion of a grid supporting the whole structure (phantom), realised as a weft of thin wires made of the same material of the phantom. This grid supports possible islands or parts of very thin chambers and thus not self-sustaining. Such grid is automatically inserted by the stereolithographer by modifying the data which have been already processed as above, and it is therefore produced contemporaneously with the phantom.

In this way, after phase C, all the information is in the right form for passing to the phase of effective production.

Although in the present example the problem has been simplified by limiting the number of compartments to three (GM, WM and CSF obtained with the external surface representing the cranium), it is clear that the method does not provide for a maximum number of tissues to be processed, and hence it is apt to represent all the involved tissues, such for example, in case of the brain,

white matter,

grey matter,

CSF,

bone (cranium),

muscles,

basal ganglia (caudate, putamen and pallidum),

vascular system,

possible pathological tissue (tumours, sclerosis plaques).

After phase C, production directly follows, through the use of a stereolithographer, obtaining a clearly anthropomorphic phantom of the brain as in FIG. 14. This brain will be then closed in a model of cranium, so as to also form the compartment for the CSF, as already said.

The fact that the phantom is anthropomorphic, or generally biomorphic, is interesting most of all when it is examined through the aforementioned classical examinations, obtaining images as the ones of FIG. 12, to be compared with the section of the phantom itself given in FIG. 13.

The particular characteristic of the phantom according to the present invention is that it may be used for both low resolution diagnostic equipments (PET and SPET) and high resolution ones, Computed Tomography (CT) and Magnetic Resonance Imaging (MRI), therefore it is the first anthropomorphic phantom usable for simulating “multimodality” studies.

The phantom according to the present invention may be filled with water and solutions containing radioisotopes for use in Nuclear Medicine (MN), or with solutions of contrast media or paramagnetic ions for use in Computerised Axial Tomography (CT) and Magnetic Resonance (MRI), respectively.

For summarising, the model lastly obtained represents a phantom having the following characteristics:

anthropomorphic,

multicompartmental,

with the separation interfaces among the component cavities, representing the various tissues, realised through stereolithographic technique,

“multimodality”, i.e. usable in MN, CT and MRI.

The phantom according to the invention, differently from the phantom by Hoffman, presents a multicompartmenting with the possibility of filling the various compartments with any liquid solutions or mixtures in order to simulate many more situations not only in MN but also in MRI and CT.

The aqueous solutions are preferably made of nickel and/or manganese and/or gadolinium, or, in nuclear medicine, solutions with radioisotopes normally used for the patient.

Moreover, the phantom results really anthropomorphic and not only in the acquired images.

The phantom according to the present invention is the unique anthropomorphic phantom contemporaneously usable in different modalities such as Nuclear Medicine, Magnetic Resonance and Computerised Axial Tomography.

Considering the ever increasing need of carrying out examinations with many modalities contemporaneously, even proved by the production of integrated equipments (CAT, Positron Emission Tomography—PET), the availability of a phantom like this would be very useful.

Furthermore, the process according to the present invention:

uses diagnostic images, thus there is no need for extra acquisitions for segmentation;

is completely automated;

is compatible with basic MRI equipments;

is implementable on low cost platforms;

comprises the possible automated segmentation of the pathological white matter (multiple sclerosis and leukaraiosis MS plaques).

The present invention has been described, by way of illustration and not by way of limitation, according to its preferred embodiments, but it should be understood that those skilled in the art can make variations and/or changes, without so departing from the related scope of protection, as defined by the following claims.

1. Process for preparing three-dimensional digital images for realising a biomorphic multicompartmental phantom, representing at least one organ and/or at least one system belonging to a living being, comprising a first phase A.1 of acquisition of images or “sequences” of the organ or of the system belonging to the living being, according to predefined acquisition parameters, forming a volumetric image defined by voxels, further comprising a phase A.2 of identification of tissues and/or tissue liquids and a phase B of selection of at least three of said tissues and/or tissue liquids, the process being characterised in that it comprises the following phases: C.1 verifying the adjacency of the voxels, so that each tissue or tissue liquid defines a connected volume representing the tissue or tissue liquid itself;
C.3 preparing an image presenting the surfaces of the volumes defined in phase C.1 according to the following sub-phases:
C.3.2 determining a number of surfaces equal to the number of tissues, such that they result internal to one another, even if partially tangent, said surfaces being the convolution of the surfaces of one or more volumes defined in phase C.1, said surfaces giving, by addition or subtraction, all the surfaces corresponding to the tissues or tissue liquids selected in phase B;
C.3.3 assigning a thickness to said surfaces, so that in the portions wherein two or more surfaces are tangent to one another the thickness is assigned to only one surface, the set of said thicknesses forming a connected volume.

2. Process according to claim 1, characterised in that phase C.1 comprises the following sub-phases:
C.1.1 selecting a voxel from the set of voxels forming the whole acquired volume;
C.1.2 comparing the selected voxel with a neighbourhood of six voxels which are connected to it through one face;
C.1.3 if another voxel of the same type (belonging to the same tissue or tissue liquid) does exist in said neighbourhood, examining the neighbourhood of this one, and so on recursively;
C.1.4 if during phase C.1.3 an island of one or more connected voxels of the type selected in phase C.1.1 is identified, which is surrounded by one or more volumes of voxels of other types, carrying out the following sub-phase:
C.1.4.1 if said island has size smaller than a predetermined threshold, assigning the voxels of said island to the tissue which is most represented in a region including the island.

3. Process according to claim 1, characterised in that it further comprises, after phase C.1.4.1, a phase C.1.4.2 wherein, according to the method of the previous phases, the existence of islands having size larger than said threshold is verified and, in the positive, one of the following sub-phases is alternatively carried out:
reassign the island to one of said tissues or tissue liquids;
connecting the island, through a channel, to one of said tissues or tissue liquids.

4. Process according to claim 1, characterised in that it further comprises a phase C.2 of smoothing the images in the three dimensions.

5. Process according to claim 1, characterised in that phase B further comprises the following phases:
B.1 eliminating all the tissues except a predetermined set of tissues;
B.2 filling the holes by assigning the corresponding voxels to at least one tissue of the predetermined set.

6. Process according to any claim 1, characterised in that it carries out, before phase C.3.2, the following phase:
C.3.1 transforming the vector representation of the voxels into the vector representation of the surfaces separating the several tissues.

7. Process according to claim 1, characterised in that the organ of the living being, the images of which are acquired in phase A.1, is the brain of a superior primate.

8. Process according to claim 7, characterised in that the organ of the living being, the images of which are acquired in phase A.1, is the brain of a human being.

9. Process according to claim 7, characterised in that during phase A.1 it is acquired a number of axial images ranging from 60 to 300, with layers having thickness ranging from 1 to 4 mm and with spacing from a centre to another one ranging from 0,5 to 2 mm, said images representing axial sections of the brain.

10. Process according to claim 9, characterised in that said images which are acquired are MRI images.

11. Process according to claim 9, characterised in that the T1-w and PD-T2-w sequences are acquired for each localization of layer.

12. Process according to claim 7, characterised in that said at least three tissues or tissue liquids selected in phase B are the grey matter, the white matter and the encephalorachidian liquid.

13. Process according to claim 7, characterised in that during phase C.3.2 a first surface containing the white matter plus the grey matter, a second surface containing only the grey matter, and a third surface representing the cranium surface are selected, the volume containing the encephalorachidian liquid and the volume containing only the white matter being obtained by subtraction between said surfaces.

14. Process according to claim 7, characterised in that phase B has a phase B.3 in which the definition of the tissues in the images under processing is corrected.

15. Process according to claim 14, characterised in that in phase B.3 the definition and the form of the basal ganglia of the brain are improved.

16. Process according to claim 1, characterised in that the image obtained from phase C.3.3 is modified so as to create channels entering the compartments/chambers corresponding to the selected tissues or tissue liquids, said channels being used for filling and emptying the phantom.

17. Apparatus for processing images starting from images of an organ of a living being, characterised in that it automatically carries out in a configurable mode phases A.1 and A.2 according to claim 1, and also phases B and C.

18. Computer program characterised in that it comprises code means adapted to execute, when running on a computer, the process according to claim 1.

19. Memory medium readable by a computer, storing a program, characterised in that the program is the computer program according to claim 18.

20. Biomorphic multicompartmental phantom, suitable for multianalytical examinations, characterised in that it is produced through a rapid prototyping device using the images processed according to the process according to claim 1, the surfaces having thickness being made of solid synthetic matter and the volumes representing the various tissues and/or tissue liquids being left empty and so forming several fillable compartments.

21. Phantom according to claim 20, characterised in that the rapid prototyping device is a stereolithographer.

22. Phantom according to claim 20, characterised in that said compartments are filled with water or solutions containing radioisotopes, for its use in Nuclear Medicine.

23. Phantom according to claim 20, characterised in that said compartments are filled with solutions of contrast media or paramagnetic ions, for use in Computerised Axial Tomography and Magnetic Resonance.

24. Phantom according to claim 20, characterised in that said compartments are filled with aqueous solutions of nickel and/or manganese and/or gadolinium.
